(19) United States
(12) Patent Application Publication
(10) Pub. No.: US 2025/0376654 A1
(43) Pub. Date: Dec. 11, 2025

(54) Cell Transfer Method And Cell Transfer Device

(71) Applicant: Terumo Kabushiki Kaisha, Tokyo (JP)

(72) Inventors: Liming LIU, Kanagawa (JP); Itaru OKUBO, Kanagawa (JP)

(21) Appl. No.: 19/310,771

(22) Filed: Aug. 26, 2025

Related U.S. Application Data
(63) Continuation of application No. PCT/JP2024/007197, filed on Feb. 28, 2024.

(30) Foreign Application Priority Data
Feb. 28, 2023 (JP) 2023-0029018

(51) Int. Cl. C12M 1/26; C12M 1/34

(52) U.S. Cl. CPC C12M 33/14; C12M 41/30

(57) ABSTRACT

A cell transfer method includes supplying a culture medium to a bioreactor using a pipe in communication with the bioreactor, where cells remain in the pipe (first circulation flow path) to transfer cells remaining in the pipe to the bioreactor; acquiring a predetermined physical quantity related to the supply of the culture medium to the bioreactor; and stopping the supply of the culture medium to the bioreactor when the physical quantity reaches a predetermined threshold value.

### CROSS-REFERENCE TO RELATED APPLICATIONS

The present application is a continuation of the International Patent Application No. PCT/JP2024/007197 filed Feb. 28, 2024, which designated the U.S. and claims the benefit of priority from Japanese Patent Application No. JP20230029018 filed Feb. 28, 2023. The entire disclosures of the above-identified applications are incorporated herein by reference.

### FIELD

The present invention relates to a cell transfer apparatus for moving cells remaining in a pipe to a bioreactor and to a cell transfer method using the same.

### BACKGROUND

Japanese Patent Application Laid-Open No. 2002-148258 discloses a culture apparatus capable of performing cell culture processing.

For example, the cell culture apparatus is connected to a bioreactor and includes a pipe through which a cell suspension flows. In order to culture cells using the cell culture apparatus, it is preferable to pack the cells remaining in the pipe into the bioreactor by transferring the cells to the bioreactor before culture. This process is referred to as a cell packing step. Optimization of the cell packing step is desired.

### SUMMARY

At least one example embodiment relates to a cell transfer method. The cell transfer method may include supplying a culture medium to a bioreactor using a pipe connected to the bioreactor where cells remain in the pipe to transfer cells remaining in the pipe to the bioreactor, acquiring a selected or predetermined physical quantity related to the supply of the culture medium to the bioreactor, and stopping the supply of the culture medium to the bioreactor when the physical quantity reaches a selected or predetermined threshold value.

In at least one example embodiment, the supply of the culture medium to the bioreactor may be stopped when the physical quantity is equal to or less than the threshold value. In at least one example embodiment, the culture medium may be supplied to the bioreactor for a selected or predetermined time period. The selected or predetermined time period may be referred to as an execution time. In at least one example embodiment, the execution time may be shortened, therefore the execution time of the cell culture processing may be optimized and also the consumption amount of the culture medium to be reduced.

In at least one example embodiment, the pipe may include a first pipe that is connected to a first port of the bioreactor and a second pipe that is connected to a second port of the bioreactor, while supplying the culture medium to the bioreactor the culture medium may be supplied to the bioreactor through the first and second pipes.

In at least one example embodiment, the supply of the culture medium to the bioreactor may be stopped the physical quantity in at least one of the first and second pipes reaches the threshold value.

In at least one example embodiment, the culture medium may be supplied to each of the first and second pipes such that a difference between a first time at which the physical quantity in the first pipe reaches the threshold value and a second time at which the physical quantity in the second pipe reaches the threshold value is equal to or less than a selected or predetermined difference.

In at least one example embodiment, the physical quantity may be acquired over time and the supply of the culture medium to each of the first pipe and the second pipe may be adjusted in response to a temporal change in the physical quantity.

In at least one example embodiment, the physical quantity may be acquired using an optical sensor.

In at least one example embodiment, the physical quantity may be at least one of an amount of transmitted light transmitted through the culture medium and an amount of scattered light scattered by the culture medium.

At least one example embodiment relates to a cell transfer apparatus. The cell transfer apparatus may include a supply unit configured to supply a culture medium to a bioreactor via a pipe connected to the bioreactor where cells remain in the pipe to transfer the cells remaining in the pipe to the bioreactor, an acquisition unit that acquires a selected or predetermined physical quantity related to the culture medium flowing into the bioreactor, and a stop unit that stops supply of the culture medium when the physical quantity reaches a selected or predetermined threshold value.

In at least one example embodiment, the pipe may include a first pipe connected to a first port of the bioreactor and a second pipe connected to a second port of the bioreactor, and the supply unit may supply the culture medium to the bioreactor through the first and second pipes.

In at least one example embodiment, the stop unit may stop the supply of the culture medium when the physical quantity in at least one of the first and second pipes reaches the threshold value.

In at least one example embodiment, the supply unit may supply the culture medium to each of the first pipe and the second pipe such that a difference between a second time at which the physical quantity in the first pipe reaches the threshold value and a second time at which the physical quantity in the second pipe reaches the threshold value is equal to or less than a selected or predetermined difference.

In at least one example embodiment, the acquisition unit may acquire the physical quantity over time, and the supply unit may adjust the supply of the culture medium to each of the first pipe and the second pipe in response to a temporal change in the physical quantity.

In at least one example embodiment, the physical quantity may be acquired using an optical sensor.

In at least one example embodiment, the physical quantity may be at least one of an amount of transmitted light transmitted through the culture medium and an amount of scattered light scattered by the culture medium.

### DRAWINGS

FIG. 1 is a schematic diagram illustrating a cell culture apparatus in accordance with at least one example embodiment.

FIG. 2 is a block diagram of a culture control apparatus of a cell culture apparatus (like the cell culture apparatus of FIG. 1) in accordance with at least one example embodiment.

FIG. 3 is a flowchart illustrating a method for cell culture processing that uses a cell culture apparatus (like the cell culture apparatus of FIG. 1) in accordance with at least one example embodiment.

FIG. 4 is a schematic diagram illustrating cell loading using a cell culture apparatus (like the cell culture apparatus of FIG. 1) in accordance with at least one example embodiment.

FIG. 5 is a schematic diagram illustrating cell stirring using a cell culture apparatus (like the cell culture apparatus of FIG. 1) in accordance with at least one example embodiment.

FIG. 6 is a schematic diagram illustrating cell packing using a cell culture apparatus (like the cell culture apparatus of FIG. 1) in accordance with at least one example embodiment.

FIG. 7 is a schematic diagram illustrating a cell culture step using a cell culture apparatus (like the cell culture apparatus of FIG. 1) in accordance with at least one example embodiment.

FIG. 8 is a flowchart illustrating cell packing using a cell culture apparatus (like the cell culture apparatus of FIG. 1) in accordance with at least one example embodiment. FIG. 9 is a time chart illustrating a temporal change in the amount of received transmitted light detected by a sensor unit.

### DETAILED DESCRIPTION

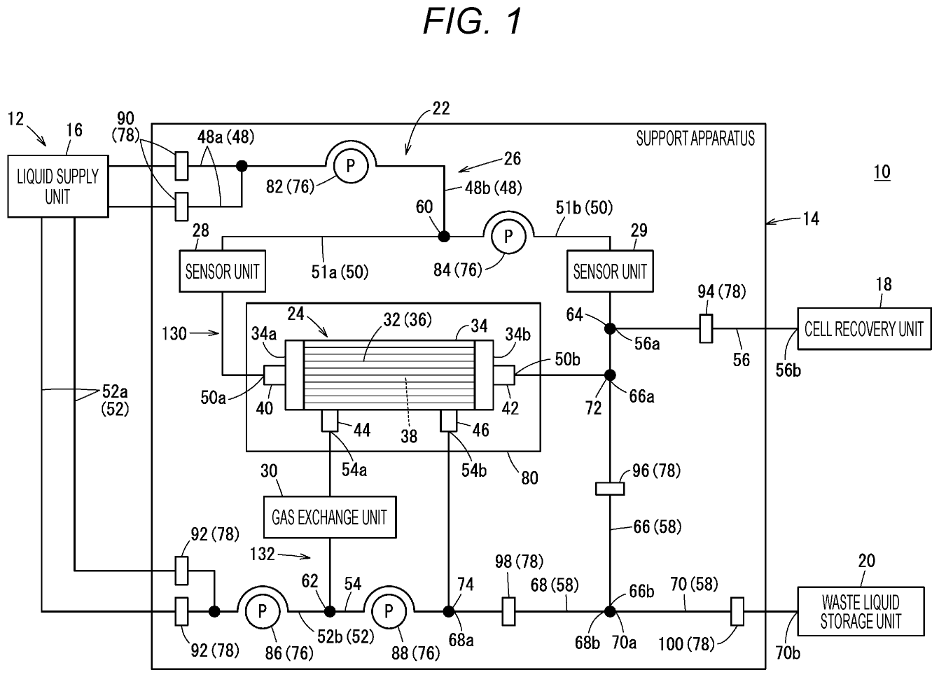

FIG. 1 is a schematic diagram illustrating a cell culture apparatus 10. The cell culture apparatus 10 cultures cells separated from biological tissue in a culture medium. The cell cultured in the cell culture apparatus 10 may include, for example, adherent cells, floating cells, or a combination of adherent cells and floating cells. The cells cultured in the cell culture apparatus 10 may include, for example, embryonic stem(ES) cells, induced pluripotent stem (iPS) cells, mesenchymal stem cells, or any combination thereof.

Liquid flows through a cell culture circuit 12. The liquid may include a cell suspension, a culture medium, a washing solution, a dissociation solution, or any combination thereof. The cell suspension is a solution including cells. The culture medium is a culture solution for growing cells. The culture medium may be selected according to the cell to be cultured. The culture medium may include, for example, a minimum essential media (MEM). The washing solution is a solution for washing the inside of the cell culture circuit 12. The washing solution may include, for example, water, a buffer solution, physiological saline, or any combination thereof. The buffer solution may include, for example, phosphate buffered salts (PBS), tris-buffered saline (TBS), or a combination of phosphate buffered salts and tris-buffered saline. The dissociation solution is a solution for detaching cells from a bioreactor 24 of the cell culture circuit 12. The dissociation solution may include a trypsin solution an ethylenediaminetetraacetic acid (EDTA) solution, or a combination of the trypsin solution and the ethylenediaminetetraacetic acid solution.

The cell culture circuit 12 may be a disposable product that is replaced for each cell culture processing. The cell culture circuit 12 may include a liquid supply unit 16, a cell recovery unit 18, a waste liquid storage unit 20, and a culture body 22.

The liquid supply unit 16 may include a first plurality of medical bags (not illustrated). Each medical bag may carry a liquid to be supplied to the culture body 22. For example, a first medical bag of the first plurality of medical bags may carry a cell suspension; a second medical bag of the first plurality of medical bags may carry a culture medium; a third medical bag of the first plurality of medical bags may carry a washing solution; and a fourth medical bag of the first plurality of medical bags may carry a dissociation solution.

The cell recovery unit 18 and the waste liquid storage unit 20 may also each include a medical bag (not illustrated), where the cell recovery unit 18 recovers the cells cultured in the culture body 22, and the waste liquid storage unit 20 stores the waste liquid generated in the culture body 22.

The culture body 22 may include the bioreactor 24, a flow path 26, sensor units 28 and 29, and a gas exchange unit 30.

The bioreactor 24 may include a plurality of hollow fiber membranes 32 and a housing 34. In at least one example embodiment, the housing 34 may have a cylindrical shape. The plurality of hollow fiber membranes 32 may be stored in the housing 34. Each hollow fiber membrane 32 may extend along the longitudinal direction of the bioreactor 24. The hollow fiber membrane 32 may be prepared using a polymer material. The hollow fiber membrane 32 may have a plurality of pores (not illustrated). A first end portion of each hollow fiber membrane 32 may be fixed to a first end portion 34a in the longitudinal direction of the housing 34. The second end portion of each hollow fiber membrane 32 may be fixed to the second end portion 34b in the longitudinal direction of the housing 34.

The bioreactor 24 may include a first region 36 and a second region 38. The first region 36 may be defined by a space inside each hollow fiber membrane 32. The second region 38 may be defined by a space between the outer peripheral surface of each hollow fiber membrane 32 and the inner peripheral surface of the housing 34. The first region 36 and the second region 38 may communicate with each other through the plurality of pores of each hollow fiber membrane 32.

The housing 34 may include a first port 40, a second port 42, a third port 44, and a fourth port 46. The first port 40 may be disposed at the first end portion 34a of the housing 34. The first port 40 may be connected to a first end portion of each hollow fiber membrane 32. The first port 40 may communicate with the first region 36. The second port 42 may be disposed at the second end portion 34b of the housing 34. The second port 42 may be connected to the second end portion of each hollow fiber membrane 32. The second port 42 may communicate with the first region 36.

The third port 44 and the fourth port 46 may be disposed on the outer peripheral surface of the housing 34. The third port 44 may be disposed between the first port 40 and a central portion of the housing 34 in the longitudinal direction. The fourth port 46 may be disposed between the second port 42 and a central portion of the housing 34 in the longitudinal direction. The third port 44 and the fourth port 46 may both communicate with the second region 38.

The flow path 26 may include a plurality of tubes (pipes) through which liquid flows. Each tube may be prepared using a soft resin material. The flow path 26 may include a first supply flow path 48, a first circulation flow path 50, a second supply flow path 52, a second circulation flow path 54, a collection flow path 56, and a waste liquid flow path 58.

The first supply flow path 48 may introduce liquids from the liquid supply unit 16 to the first circulation flow path 50. The first supply flow path 48 may include a plurality of first upstream flow paths 48a and one first downstream flow path 48b. One first upstream flow path 48a may be provided for one medical bag of the liquid supply unit 16. The first upstream flow path 48a may be connected to the medical bag of the liquid supply unit 16. Each of the first upstream flow paths 48a may be connected to the first downstream flow path 48b. The first downstream flow path 48b may be connected to a first merging portion 60 of the first circulation flow path 50.

The first circulation flow path 50 may introduce the liquid introduced from the first supply flow path 48 to the bioreactor 24. In addition, the first circulation flow path 50 may introduce the liquid discharged from the bioreactor 24 to the bioreactor 24 again. The first end portion 50a of the first circulation flow path 50 may be connected to the first port 40 of the bioreactor 24. The second end portion 50b of the first circulation flow path 50 may be connected to the second port 42 of the bioreactor 24. The first circulation flow path 50 may communicate with an inner hole (first region 36) of each hollow fiber membrane 32. The first merging portion 60 may be disposed in the first circulation flow path 50. In the first circulation flow path 50, a portion partitioned by the first end portion 50a and the first merging portion 60 may be referred to as a first flow path 51a (first pipe). In the first circulation flow path 50, a portion partitioned by the second end portion 50b and the first merging portion 60 may be referred to as a second flow path 51b (second pipe). A collection branch portion 64 may be disposed in the second flow path 51b. Furthermore, in the second flow path 51b, a first branch portion 72 may be disposed between the collection branch portion 64 and the second end portion 50b.

The second supply flow path 52 may introduce various liquids of the liquid supply unit 16 to the second circulation flow path 54. The second supply flow path 52 may include a plurality of second upstream flow paths 52a and one second downstream flow path 52b. One second upstream flow path 52a may be provided for one medical bag of the liquid supply unit 16. The second upstream flow path 52a may be connected to the medical bag of the liquid supply unit 16. In addition, each of the second upstream flow paths 52a may be connected to the second downstream flow path 52b. The second downstream flow path 52b may be connected to a second merging portion 62 of the second circulation flow path 54.

The second circulation flow path 54 may introduce the liquid introduced from the second supply flow path 52 to the bioreactor 24. In addition, the second circulation flow path 54 may introduce the liquid discharged from the bioreactor 24 to the bioreactor 24 again. The first end portion 54a of the second circulation flow path 54 may be connected to the third port 44 of the bioreactor 24. A second end portion 54b of the second circulation flow path 54 may be connected to the fourth port 46 of the bioreactor 24. The second circulation flow path 54 may communicate with a space (second region 38) between the plurality of hollow fiber membranes 32 and the housing 34. The second merging portion 62 may be disposed in the second circulation flow path 54. In addition, in the second circulation flow path 54, a second branch portion 74 may be disposed between the second merging portion 62 and the second end portion 54b.

The collection flow path 56 may introduce the cell suspension discharged from the bioreactor 24 into the cell recovery unit 18. The collection flow path 56 may branch from the first circulation flow path 50. A first end portion 56a of the collection flow path 56 may be connected to the collection branch portion 64 of the first circulation flow path 50. A second end portion 56b of the collection flow path 56 may be connected to the medical bag of the cell recovery unit 18.

The waste liquid flow path 58 may introduce the liquid in the first circulation flow path 50 and the second circulation flow path 54 into the waste liquid storage unit 20. The waste liquid flow path 58 may include a first waste liquid flow path 66, a second waste liquid flow path 68, and a third waste liquid flow path 70. The first waste liquid flow path 66 may branch from the first circulation flow path 50. A first end portion 66a of the first waste liquid flow path 66 may be connected to the first branch portion 72 of the first circulation flow path 50. The second waste liquid flow path 68 may branch from the second circulation flow path 54. A first end portion 68a of the second waste liquid flow path 68 may be connected to the second branch portion 74 of the second circulation flow path 54. The second end portion 66b of the first waste liquid flow path 66 and the second end portion 68b of the second waste liquid flow path 68 may be connected to a first end portion 70a of the third waste liquid flow path 70. A second end portion 70b of the third waste liquid flow path 70 may be connected to the medical bag of the waste liquid storage unit 20.

The sensor unit 28 may be disposed in the first flow path 51a. On the other hand, the sensor unit 29 may be disposed in the second flow path 51b. The sensor unit 28 may detect a selected or predetermined physical quantity related to the liquid flowing through the first flow path 51a. The sensor unit 29 may detect a selected or predetermined physical quantity related to the liquid flowing through the second flow path 51b. The selected or predetermined physical quantity may be a physical quantity proportional to the number of cells in the liquid. For example, each of the sensor units 28 and 29 may include a light source and one or more optical sensors (light receivers). The light source may irradiate the liquid with light. The optical sensor may receive transmitted light transmitted through the liquid. The optical sensor may output an electric signal corresponding to the amount of received light to a controller 112. The optical sensor may receive scattered light (forward scattered light, side scattered light, backscattered light, and the like) instead of the transmitted light. In addition, each of the sensor units 28 and 29 may include a sensor (for example, a dielectric constant sensor) including two or more electrodes instead of the light source and the optical sensor.

The gas exchange unit 30 may be disposed between the second merging portion 62 and the third port 44 in the second circulation flow path 54. The gas exchange unit 30 may suppl a gas of a selected or predetermined component to the liquid (culture medium) flowing through the second circulation flow path 54. The gas used in the gas exchange unit 30 may have, for example, a component close to air. That is, the gas may include nitrogen, oxygen, and carbon dioxide.

The cell culture circuit 12 may be detachable from the support apparatus 14. The support apparatus 14 may include a cassette that supports the cell culture circuit 12. The support apparatus 14 may be a reusable product that can be used a plurality of times.

The support apparatus 14 may include a plurality of pumps 76, a plurality of clamps 78, and a reactor driving unit 80. Each of the plurality of pumps 76, the plurality of clamps 78, and the reactor driving unit 80 may include an electric actuator. Each of the plurality of pumps 76, the plurality of clamps 78, and the reactor driving unit 80 may include a fluid actuator.

Each pump 76 can apply a flow force to the liquid in the flow path 26, for example, by squeezing the tube forming the flow path 26. Each pump 76 may be operated by power supplied from a pump drive circuit 114.

The plurality of pumps 76 may include a first supply pump 82, a first circulation pump 84, a second supply pump 86, and a second circulation pump 88. Each pump of the plurality of pumps 76 may function as follows. As illustrated in FIG. 1, a state in which the cell culture circuit 12 is attached to the support apparatus 14 may be referred to as a “set state”.

In the set state, the first downstream flow path 48b may be attached to the first supply pump 82. The first supply pump 82 may be configured to apply a flow force in a direction from the liquid supply unit 16 toward the first circulation flow path 50 to the liquid in the first supply flow path 48.

In the set state, the second flow path 51b of the first circulation flow path 50 may be attached to the first circulation pump 84. The first circulation pump 84 may be configured to apply a flow force in a direction from the second port 42 to the first port 40 to the liquid in the first circulation flow path 50. The first circulation pump 84 may also be configured to apply a flow force in a direction from the first port 40 to the second port 42 to the liquid in the first circulation flow path 50.

In the set state, the second downstream flow path 52b may be attached to the second supply pump 86. The second supply pump 86 may be configured to apply a flow force in a direction from the liquid supply unit 16 toward the second circulation flow path 54 to the liquid in the second supply flow path 52.

In the set state, the second circulation flow path 54 may be attached to the second circulation pump 88. The second circulation pump 88 may be configured to apply a flow force in a direction from the fourth port 46 to the third port 44 to the liquid in the second circulation flow path 54. The second circulation pump 88 may also apply a flow force in a direction from the third port 44 to the fourth port 46 to the liquid in the second circulation flow path 54.

Each clamp 78 may close the flow path 26 by compressing the tube forming the flow path 26 in the lateral direction. Each clamp 78 may function as an on-off valve. Each clamp 78 may be operated using power supplied from a clamp drive circuit 116.

The plurality of clamps 78 may include a plurality of first supply clamps 90, a plurality of second supply clamps 92, a collection clamp 94, a first waste liquid clamp 96, a second waste liquid clamp 98, and a third waste liquid clamp 100. Each clamp 78 may function as follows.

In the set state, one first upstream flow path 48a may be attached to one first supply clamp 90. In other words, each of the first upstream flow paths 48a may be supported by any one of the first supply clamps 90. The first supply clamp 90 may open and close the first supply flow path 48.

In the set state, one second upstream flow path 52a may be attached to one second supply clamp 92. In other words, each of the second upstream flow paths 52a may be supported by any one of the second supply clamps 92. The second supply clamp 92 may open and close the second supply flow path 52.

In the set state, the collection flow path 56 may be attached to the collection clamp 94. The collection clamp 94 may open and close the collection flow path 56. In the set state, the first waste liquid flow path 66 may be attached to the first waste liquid clamp 96. The first waste liquid clamp 96 may open and close the first waste liquid flow path 66. In the set state, the second waste liquid flow path 68 may be attached to the second waste liquid clamp 98. The second waste liquid clamp 98 may open and close the second waste liquid flow path 68. In the set state, the third waste liquid flow path 70 may be attached to the third waste liquid clamp 100. The third waste liquid clamp 100 may open and close the third waste liquid flow path 70.

The reactor driving unit 80 may support the bioreactor 24. In addition, the reactor driving unit 80 may rotate the bioreactor 24 in one direction and the opposite direction about an axis orthogonal to the longitudinal direction of the bioreactor 24. The reactor driving unit 80 may be operated by power supplied from a reactor drive circuit 118.

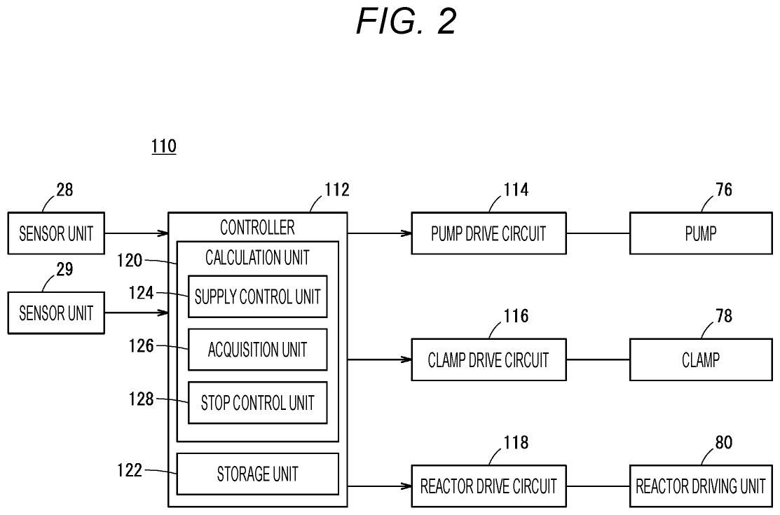

FIG. 2 is a block diagram of a culture control apparatus 110 included in the cell culture apparatus 10. The culture control apparatus 110 may control each of the plurality of pumps 76, the plurality of clamps 78, and the reactor driving unit 80. The culture control apparatus 110 may function as a cell transfer apparatus that transfers the cells remaining in the first circulation flow path 50 to the bioreactor 24.

The culture control apparatus 110 may include the sensor units 28 and 29, the controller 112, the pump drive circuit 114, the clamp drive circuit 116, and the reactor drive circuit 118. In at least one example embodiment, a computer may be used as the controller 112. The controller 112 may include a calculation unit 120 and a storage unit 122.

The calculation unit 120 may be configured by a processor. The processor may include, for example, a central processing unit (CPU), a graphics processing unit (GPU), or a combination of the central processing unit and the graphics processing unit. In at least one example embodiment, the calculation unit 120 may be configured by a processing circuit.

The calculation unit 120 may include a supply control unit 124 (supply unit), an acquisition unit 126, and a stop control unit 128 (stop unit). Each of the supply control unit 124, the acquisition unit 126, and the stop control unit 128 maybe realized by executing a program or programs by the calculation unit 120. The program or programs may be stored in the storage unit 122.

At least a part of the supply control unit 124, the acquisition unit 126, and the stop control unit 128 may be realized by an integrated circuit. The integrated circuit may include an application specific integrated circuit (ASIC), a field-programmable gate array (FPGA), or a combination of the application specific integrated circuit and the field-programmable gate array. Furthermore, at least a part of the supply control unit 124, the acquisition unit 126, and the stop control unit 128 may be configured by an electronic circuit. The electronic circuit may include a discrete device.

The supply control unit 124 may be configured to perform various controls related to supply of liquid from the liquid supply unit 16 to the bioreactor 24. The acquisition unit 126 may be configured to acquire the amount of received transmitted light (or the amount of received scattered light) over time on the basis of the electrical signals output from the sensor units 28 and 29. The stop control unit 128 may be configured to perform various types of control related to stop of supply of liquid from the liquid supply unit 16 to the bioreactor 24.

The storage unit 122 may include a volatile memory (not illustrated), a non-volatile memory (not illustrated), or a combination of the volatile memory and the non-volatile memory. The volatile memory may include, for example, a random access memory (RAM). The volatile memory may be used as a working memory of the processor. For example, the volatile memory may temporarily store data for processing or operation. The non-volatile memory may include a read only memory (ROM), a flash memory, of a combination of the read only memory and the flash memory. The non-volatile memory may be used as a memory for storage. For example, the non-volatile memory may store a program, a table, a map, or any combination thereof. At least a part of the storage unit 122 may be provided in the processor, the integrated circuit, or a combination of the processor and the integrated circuit.

The pump drive circuit 114 may be configured to supply power to the actuator of the pump 76 such that the pump 76 may be operated according to a pump operation signal output from the controller 112. Each clamp drive circuit 116 may be configured to supply power to the actuator of the clamp 78 such that the clamp 78 may be operated according to the clamp operation signal output from the controller 112. The reactor drive circuit 118 may be configured to supply power to the actuator of the reactor driving unit 80 in response to a reactor operation signal output from the controller 112.

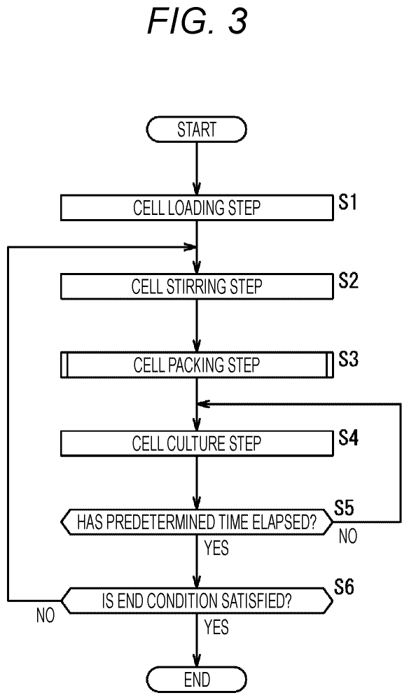

FIG. 3 is a flowchart illustrating a method for cell culture processing. The user may use the input apparatus to start the cell culture processing. The calculation unit 120 may be configured to start cell culture processing as illustrated in FIG. 3 in response to the start operation.

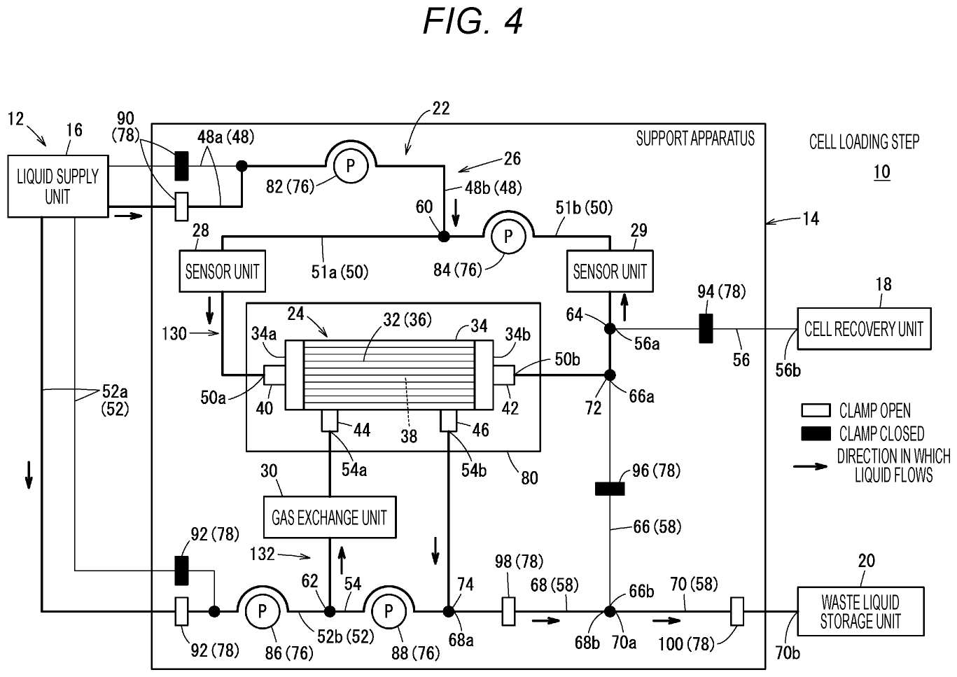

At step S1, the calculation unit 120 may be configured to perform cell loading. Prior to and/or during the cell loading, the supply control unit 124 may be configured to output a pump operation signal corresponding with the cell loading to the pump drive circuit 114. Prior to and/or during the cell loading, the supply control unit 124 may be configured to output a clamp operation signal corresponding with the cell loading to the clamp drive circuit 116. In at least one example embodiment, the output of at least one of the pump operation signal and the clamp operation signal may initiate the cell loading. During and/or after the output of at least one of the pump operation signal corresponding with cell loading and the clamp operation signal corresponding with cell loading, the cell culture apparatus 10 may enter the state as illustrated in FIG. 4 during which a cell suspension supported by the medical bag of the liquid supply unit 16 may flow through the first supply flow path 48 and the first circulation flow path 50 and may be supplied to (or introduced into) the bioreactor 24. That is, the cells may be introduced into the first region 36 (inside the hollow fiber membrane 32) of the bioreactor 24. The stop control unit 128 may be configured to stop the cell loading step at a selected or predetermined timing. When step S1 ends, the processing may proceed to step S2.

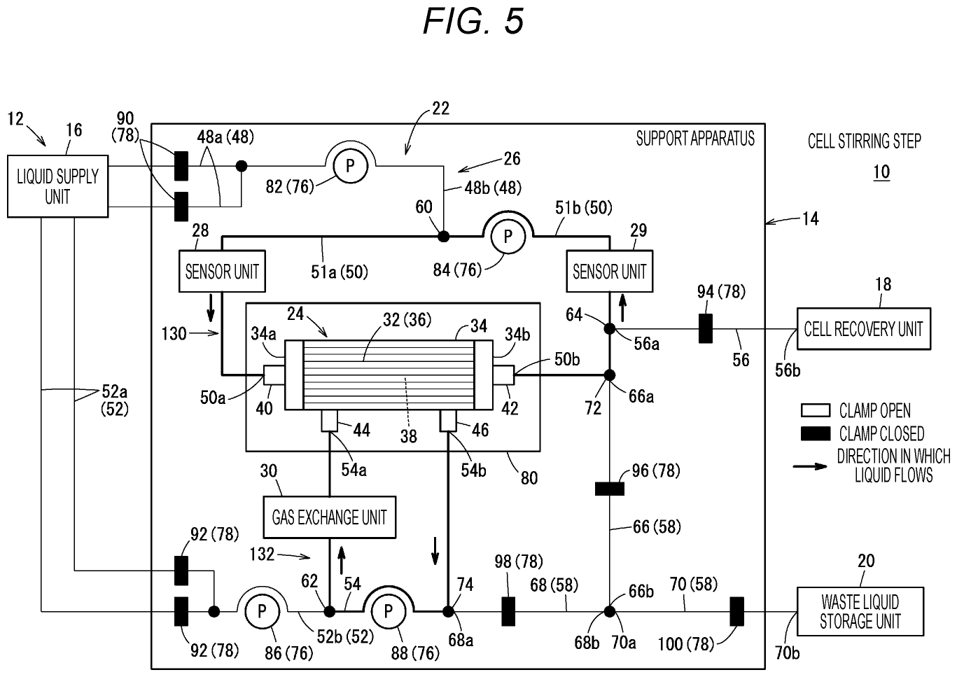

With renewed reference to FIG. 3, at step S2, the calculation unit 120 may be configured to perform a cell stirring. Prior to and/or during the cell stirring, the supply control unit 124 may be configured to output pump operation signal corresponding with cell stirring to the pump drive circuit 114. Prior to and/or during the cell stirring, the supply control unit 124 may be configured to output a clamp operation signal corresponding with cell stirring to the clamp drive circuit 116. In at least one example embodiment, the output of at least one of the pump operation signal corresponding with cell stirring and/or the clamp operation signal corresponding with cell stirring may initiate the cell stirring. During and/or after the output of at least one of the pump operation signal corresponding with cell stirring and/or the clamp operation signal corresponding with cell stirring, the cell culture apparatus 10 may enter the state illustrated in FIG. 5 during which the cell suspension may circulate in a circulation path 130 composed of the bioreactor 24 and the first circulation flow path 50 causing the cell suspension to be stirred and the cells to be diffused within the cell suspension. A state in which cells are uniformly diffused in the cell suspension may be referred to as a uniform diffusion state. In a uniform diffusion state, nutrients are evenly distributed among the cells. The supply control unit 124 may output a reactor operation signal for operating the reactor driving unit 80 to the reactor drive circuit 118. The reactor driving unit 80 may alternately rotates the bioreactor 24 in one direction and in the opposite direction according to the reactor operation signal, which may allow the cell suspension to be homogeneously diffused more quickly. The stop control unit 128 may be configured to stop the cell stirring when a selected or predetermined stirring time has elapsed since the start of the cell stirring. When step S2 ends, the processing may proceed to step S3.

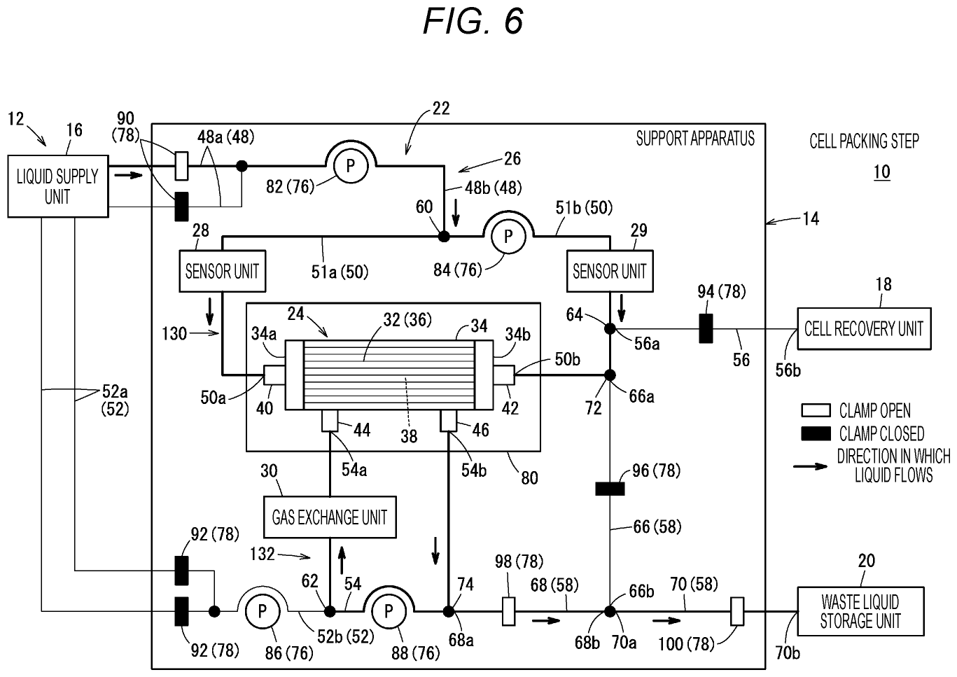

At step S3, the calculation unit 120 may be configured to perform cell packing. Prior to and/or during the cell packing, the supply control unit 124 may be configured to output a pump operation signal corresponding with cell packing to the pump drive circuit 114. Prior to and/or during the cell packing, the supply control unit 124 may be configured to output a clamp operation signal corresponding with cell packing to the clamp drive circuit 116. In at least one example embodiment, the output of at least one of pump operation signal corresponding with cell packing and the clamp operation signal corresponding with cell packing may initiated the cell packing. During and/or after the output of at least one of the pump operation signal corresponding with cell packing and the clamp operation signal corresponding with cell packing, the cell culture apparatus 10 may enter the state illustrated in FIG. 6 during which the culture medium as supported by the medical bag of the liquid supply unit 16 may flow through the first supply flow path 48 and the first flow path 51a to be supplied to (or introduced into) the bioreactor 24. The culture medium as supported by the medical bag of the liquid supply unit 16 may also through the first supply flow path 48 and the second flow path 51b to be supplied (or introduced into) to the bioreactor 24. As a result, the cells remaining in the first circulation flow path 50 may move to the first region 36 (inside the hollow fiber membrane 32) of the bioreactor 24. The culture medium may also circulate in a circulation path 132 including the bioreactor 24 and the second circulation flow path 54. The excess culture medium may be discharged to the waste liquid storage unit 20. When step S3 ends, the processing may proceed to step S4.

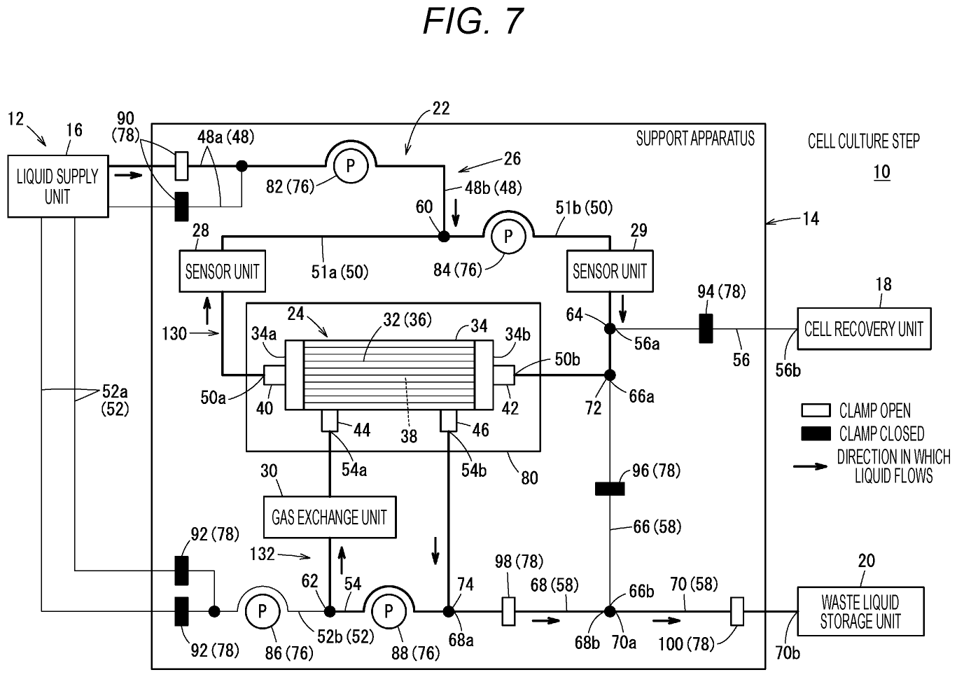

At step S4, the calculation unit 120 may be configured to perform a cell culturing. Prior to and/or during the cell culturing, the supply control unit 124 may be configured to output a pump operation signal corresponding with cell culturing to the pump drive circuit 114. Prior to and/or during the cell culturing, the supply control unit 124 may be configured to output a clamp operation signal corresponding with cell culture to the clamp drive circuit 116. In at least one example embodiment, the output of at least one of the pump operation signal corresponding with cell culturing and the clamp operation signal corresponding with cell culturing may initiated the cell culturing. During and/or after the output of at least one of the pump operation signal corresponding with cell culturing and the clamp operation signal corresponding with cell culturing, the cell culture apparatus 10 may enter the state illustrated in FIG. 7 during which the culture medium supported by the medical bag of the liquid supply unit 16 may flow through the first supply flow path 48 and the second flow path 51b and may be supplied to (or introduced into) the first region 36 (inside the hollow fiber membrane 32) of the bioreactor 24. The culture medium may also circulate in the circulation path 130 including the bioreactor 24 and the first circulation flow path 50 to provide nutrients to the cells. The culture medium may also circulate in the circulation path 132 including the bioreactor 24 and the second circulation flow path 54. The excess culture medium may be discharged to the waste liquid storage unit 20.

At step S5, the stop control unit 128 may be configured to determine whether a selected to predetermined culture time has elapsed since the start of the cell culture step. In at least one example embodiment, the storage unit 122 may be configured to store the predetermined culture time in advance. When the selected or predetermined culture time has elapsed (step S5: YES), the processing may proceed to step S6. When the selected or predetermined time has not elapsed (step S5: NO), the cell culturing of step S4 may be continuously performed.

When the processing proceeds from step S5 to step S6, the stop control unit 128 may be configured to determine whether the end condition of the series of cell culture processing as illustrated in FIG. 3 is satisfied. The storage unit 122 may be configured to store a calibration curve that indicate the relationship between the amount of received transmitted light (or scattered light) of the cell suspension and the number of cells. The storage unit 122 may also be configured to store a threshold value as an end condition in advance. The stop control unit 128 may be configured to estimate the number of cells contained in the cell suspension on the basis of the amount of light received by the sensor unit 28 and the calibration curve. The stop control unit 128 may be configured to determine that the end condition is satisfied in a case where the estimated number of cells is equal to or greater than the threshold value. The storage unit 122 may be configured to store a calibration curve indicating the relationship between the amount of received transmitted light (or scattered light) of the cell suspension and the cell concentration. When the estimated number of cells is equal to or larger than the threshold value (step S6: YES), the series of cell culture processing may be ended. When the estimated number of cells is less than the threshold value (step S6: NO), the processing may return to the cell stirring of step S2.

When the cell culture processing illustrated in FIG. 3 is completed, the supply control unit 124 may be configured to supply the dissociation solution to the bioreactor 24 and to transfer the cells inside the bioreactor 24 to the cell recovery unit 18.

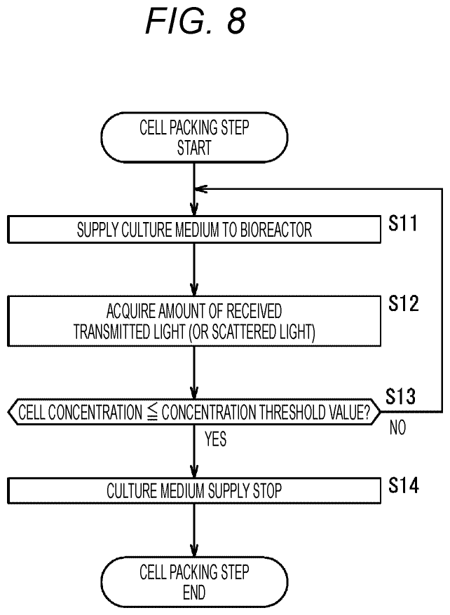

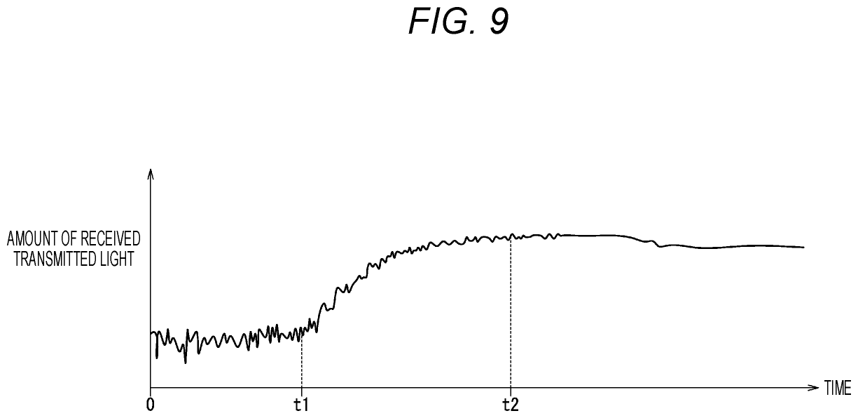

FIG. 8 is a flowchart illustrating a method for cell packing. After step S2 illustrated in FIG. 3, cell packing may be performed.

At step S11 (supply step), the supply control unit 124 may be configured to control the first supply pump 82 and the first circulation pump 84 to supply the culture medium to the bioreactor 24 via the first supply flow path 48 and the first flow path 51a. The supply control unit 124 may also be configured to control the first supply pump 82 and the first circulation pump 84 to supply the culture medium to the bioreactor 24 via the first supply flow path 48 and the second flow path 51b. The cells remaining in the first flow path 51a may move to the bioreactor 24 together with the culture medium. The cells remaining in the second flow path 51b may move to the bioreactor 24 together with the culture medium. Then, each of the cell concentration in the first flow path 51a and the cell concentration in the second flow path 51b may decrease.

The timing at which the cell concentration in the first flow path 51a becomes equal to or less than a selected or predetermined concentration threshold value may be referred to as first timing. In addition, the timing at which the cell concentration in the second flow path 51b becomes equal to or less than the selected or predetermined concentration threshold value may be referred to as second timing. The supply control unit 124 may be configured to control the first supply pump 82 and the first circulation pump 84 such that the difference between the first timing and the second timing is equal to or less than a predetermined difference. Accordingly, the cell packing can be completed in the shortest time. For example, the ratio between the flow rate of the culture medium supplied from the first supply flow path 48 to the first flow path 51a and the flow rate of the culture medium supplied from the first supply flow path 48 to the second flow path 51b may match the ratio between the volume of the first flow path 51a and the volume of the second flow path 51b. The storage unit 122 may be configured to store the target flow rate of the first supply pump 82 and the target flow rate of the first circulation pump 84. The supply control unit 124 may be configured to control each of the first supply pump 82 and the first circulation pump 84 to achieve the target flow rates stored in the storage unit 122. In at least one example embodiment, it may not be essential to set the difference between the first timing and the second timing to a predetermined difference or less. For example, the difference between the first timing and the second timing may be larger than the predetermined difference.

At step S12 (acquisition step), the acquisition unit 126 may be configured to acquire the amount of received transmitted light on the basis of the electric signal output from each of the sensor units 28 and 29. The acquisition unit 126 may be configured to acquire the amount of received scattered light instead of the amount of received transmitted light. The acquisition unit 126 may be configured to acquire a received light amount of each transmitted light every predetermined sampling time.

At step S13, the stop control unit 128 may be configured to calculate the cell concentration of the culture medium in the first flow path 51a on the basis of the amount of received transmitted light (or scattered light) detected by the sensor unit 28 and the calibration curve. Similarly, the stop control unit 128 may be configured to calculate the cell concentration of the culture medium in the second flow path 51b on the basis of the amount of received transmitted light (or scattered light) detected by the sensor unit 29 and the calibration curve. Furthermore, the stop control unit 128 may be configured to compare each calculated cell concentration with a predetermined concentration threshold value. The calibration curve may indicate the relationship between the amount of received light and the cell concentration. As the concentration threshold value, an upper limit value of the allowable cell concentration may be set. Each of the calibration curve and the concentration threshold value may be stored in advance in the storage unit 122. When each cell concentration is equal to or less than the concentration threshold value (step S13: YES), the processing may proceed to step S14. When at least one of the cell concentrations is higher than the concentration threshold value (step S13: NO), the processing may return to step S11 and the processing of step S11 may be continued.

When the step proceeds from step S13 to step S14 (stop step), the stop control unit 128 may be configured to stop the supply of the culture medium to the bioreactor 24. That is, the stop control unit 128 may be configured to stop the first supply pump 82 and the first circulation pump 84. Upon completion of step S14, the processing may proceed to the cell culturing (step S4) illustrated in FIG. 3.

In at least one example embodiment, in step S13, the stop control unit 128 may be configured to convert the received light amount into the cell concentration and to perform the stop determination on the basis of the comparison result between the cell concentration and the concentration threshold value. Alternatively, the stop control unit 128 may be configured to perform the stop determination on the basis of the comparison result between the amount of received light and the light threshold value. There may be a correlation between the amount of received light and the cell concentration. As the amount of received transmitted light increases, the cell concentration decreases. On the other hand, as the amount of received scattered light increases, the cell concentration increases.

FIG. 9 is a time chart illustrating a temporal change in the amount of received transmitted light detected by the sensor unit 28. The temporal change of the amount of received transmitted light detected by the sensor unit 29 may also be similar to that in FIG. 9. As illustrated in FIG. 9, the amount of received transmitted light gradually increases from the time point t1 at which the cell packing step is started. That is, by executing the cell packing, the number of cells remaining in the first flow path 51a may decrease.

Then, the turbidity of the culture medium in the first flow path 51a may decrease and the amount of received transmitted light may increase. The increase amount per unit time of the amount of received light may gradually increase with the lapse of time. The increase amount per unit time of the amount of received light may gradually decrease after increasing to some extent and may approach zero at time point t2. When the increase amount of the amount of received light per unit time approaches zero it is understood most of the cells in the first flow path 51a have moved to the bioreactor 24.

The stop control unit 128 may be configured to stop the supply of the culture medium when the cell concentration (physical quantity) becomes equal to or less than the concentration threshold value (e.g., selected or predetermined threshold value). The cell packing may be performed only for a necessary minimum time. The execution time of the cell packing step may be shortened and the execution time of the cell culture processing illustrated in FIG. 3 may be shortened. Therefore, the cell culture processing may be optimized. In addition, the consumption amount of the culture medium may be reduced by shortening the cell packing time.

In at least one example embodiment, only one of the sensor units 28 and 29 may be provided in the cell culture circuit 12. In such instances, in step S11, the supply control unit 124 may be configured to control the first supply pump 82 and the first circulation pump 84 so that the first timing at which the cell concentration in the first flow path 51a becomes equal to or less than the predetermined concentration threshold value and the second timing at which the cell concentration in the second flow path 51b becomes equal to or less than the selected or predetermined concentration threshold value are substantially the same.

It should be appreciated that the present invention is not limited to the above disclosure and various configurations can be adopted without departing from the gist of the present invention.

1. A method comprising:
causing a culture medium to flow into a bioreactor via a pipe connected to the bioreactor to transfer cells remaining in the pipe to the bioreactor;
acquiring a physical quantity related to the flow of the culture medium into the bioreactor via the pipe; and
stopping flow of the culture medium into the bioreactor via the pipe when the physical quantity in the pipe reaches a threshold.

2. The method of claim 1, wherein the physical quantity is a predetermined physical quantity and the threshold is a predetermined threshold.

3. The method of to claim 1, wherein
the pipe includes:
a first pipe connected to a first port of the bioreactor, and
a second pipe connected to a second port of the bioreactor; and
the culture medium being flown into the bioreactor through both the first and second pipes.

4. The method of claim 3, wherein
the physical quantity is a first physical quantity of the first pipe;
the threshold is a first threshold;
the method further includes:
acquiring a second physical quantity related to the flow of the culture medium through the second pipe; and
stopping flow of the culture medium into the bioreactor via the second pipe when the second physical quantity in the second pipe reaches the second threshold

5. The method of claim 4, wherein the first physical quantity is the same type as the second physical quantity and the first threshold is the same value as the second threshold.

6. The method of claim 4, wherein
the culture medium is supplied to each of the first pipe and the second pipe such that a difference between a timing at which the first physical quantity in the first pipe reaches the first threshold and a timing at which the second physical quantity in the second pipe reaches the second threshold is equal to or less than a selected or predetermined difference.

7. The method of claim 3, wherein
the first pipe is connected to a first region of the bioreactor,
the second pipe is connected to a second region of the bioreactor,
the first region communicates with the second region via a plurality of pores in a membrane defining the first and second regions of the bioreactor.

8. The method of claim 1, wherein
the physical quantity is acquired over time, and
the method further includes:
adjusting the flow of the culture medium into the bioreactor via the pipe in response to a temporal change in the physical quantity.

9. The method of claim 1, wherein the physical quantity is acquired using an optical sensor.

10. The method of claim 9, wherein the physical quantity includes at least one of an amount of transmitted light transmitted through the culture medium and an amount of scattered light scattered by the culture medium.

11. An apparatus comprising:
a supply unit that is configured to cause a culture medium to flow into a bioreactor via a pipe connected to the bioreactor to transfer cells remaining in the pipe to the bioreactor;
an acquisition unit that is configured to acquires a physical quantity related to the flow of the culture medium into the bioreactor via the pipe; and
a stop unit that is configured to stop the flow of the culture medium into the bioreactor via the pipe when the physical quantity reaches a threshold value.

12. The apparatus of claim 11, wherein the physical quantity is a predetermined physical quantity and the threshold is a predetermined threshold.

13. The apparatus of claim 11, wherein
the pipe includes:
a first pipe connected to a first port of the bioreactor, and
a second pipe connected to a second port of the bioreactor; and
the supply unit is configured to cause the culture medium to flow into the bioreactor through both the first and second pipes.

14. The apparatus of claim 13, wherein
the physical quantity is a first physical quantity of the first pipe;
the threshold is a first threshold; and
the stop unit is further configured to stop the flow of the culture medium into the bioreactor via the second pipe when the second physical quantity in the second pipe reaches a second threshold.

15. The apparatus of claim 14, wherein the first physical quantity is the same type as the second physical quantity and the first threshold is the same value as the second threshold.

16. The apparatus of claim 14, wherein the supply unit is configured to supply the culture medium to each of the first pipe and the second pipe such that a difference between a timing at which the first physical quantity in the first pipe reaches the first threshold and a timing at which the second physical quantity in the second pipe reaches the second threshold is equal to or less than a selected or predetermined difference.

17. The apparatus of claim 13, wherein
the first pipe is connected to a first region of the bioreactor,
the second pipe is connected to a second region of the bioreactor,
the first region communicates with the second region via a plurality of pores in a membrane defining the first and second regions of the bioreactor.

18. The apparatus of claim 11, wherein
the acquisition unit is configured to acquire the physical quantity over time, and
the supply unit is configured to adjust the flow of the culture medium into the bioreactor via the pipe in response to a temporal change in the physical quantity.

19. The apparatus of claim 11, wherein the physical quantity is acquired using an optical sensor.

20. The apparatus of claim 13, wherein
the physical quantity includes at least one of an amount of transmitted light transmitted through the culture medium and an amount of scattered light scattered by the culture medium.
